(19) United States
(12) Plant Patent Application Publication
(10) Pub. No.: US 2010/0146677 P1
(43) Pub. Date: Jun. 10, 2010

(54) Agapanthus africanus cv. ponto's queen of the nile

(50) ;

(75) Inventor: William H. Ponto, Vista, CA (US)

Correspondence Address: THE MAXHAM FIRM, 9330 SCRANTON ROAD, SUITE 350, SAN DIEGO, CA, 92121, US

(73) Assignee: PONTO NURSERY, INC., Vista, CA (US)

(21) Appl. No.: 12/315,941

(22) Filed: Dec. 9, 2008

(51) Int. Cl. A01H 5/00

(52) U.S. Cl. PLT/313

(57) ABSTRACT

A new and distinct selection of Agapanthus africanus (commonly known as Lily of the Nile) which herein is referred to as Agapanthus africanus cv. Ponto's Queen of the Nile. Ponto's Queen of the Nile differs from its parent plant, Agapanthus africanus Queen Ann, by possessing a gold leaf with a dark green stripe in the middle rather than an all-green leaf. The flower stock is green with a gold stripe as well.

### DISCOVERY

This invention relates to a new and distinct selection of Agapanthus africanus Queen Ann, a member of the Alliancae family. Agapanthus is commonly known as Lily of the Nile. Agapanthus africanus cv. Ponto's Queen of the Nile, this new variety, originated as a selected seedling from Agapanthus africanus Queen Ann, a self pollinating, non patent plant. The discovery was made by Bill Ponto, at Ponto Nursery, located at 2545 Ramona Drive, Vista, Calif. 92084, in the spring of 1998. The plant to date is known to exist only within the boundaries of Ponto Nursery, and has not been described in any publication.

This distinctive plant was selected out, allowed to develop and mature, and then asexually reproduced by divisions. Each of the progeny exhibit identical characteristics to the original selected prototype, establishing this variety as stable and true to type when produced by asexual means.

### PROPAGATION

This new cultivar has been strictly asexually reproduced by divisions only within the boundaries of this location at Ponto Nursery. Sexual reproduction such as seed propagation would not produce a true form of this cultivar and would result in loss of selected attributes. The divisions have been asexually reproduced for the last ten years, thereby establishing this cultivar as a new and distinctive plant. This plant is unknown in any written publication found.

### BRIEF SUMMARY OF THE INVENTION

The new variety is consistent with its parent plant, Agapanthus africanus Queen Ann in regards to hardiness. It appears to be hardy to 29° F. The flower is typical of the parent plant, blooming in late spring and early summer. The flower longevity is one to three weeks.

This new variety, Agapanthus africanus cv. Ponto's Queen of the Nile, differs from its' parent plant, Agapanthus africanus Queen Ann, by a characteristic gold leaf with a dark green stripe in the middle, rather than an all-green leaf. The flower stock, or peduncle, is green with a gold stripe as well.

This new variety would make an excellent border for a lawn. It can also be used in flower beds as well as a container plant for the patio. This plant is suitable for use as an annual when temperatures dip below 29° F. and a perennial in all other areas.

### DISEASE AND INSECTS

There were no known pathogens or insects observed that were not typical of any Agapanthus africanus.

### BRIEF DESCRIPTION OF THE PHOTOGRAPHS

The accompanying photographs show a typical specimen of the plant according to this invention in full color, demonstrating the unique and distinctive coloration of the leaves and flower stock or peduncle, the color rendition being as nearly true as it is possible to obtain by conventional procedures, in which:

FIG. 1 shows the complete plant of the new variety in two five-gallon containers at 4 years of age;

FIG. 2 shows many of the plants of this new variety, revealing the uniformity of the foliage;

FIG. 3 shows the color of the inflorescence and its relationship to the foliage;

FIG. 4 shows a greenhouse full of the new variety in a multiplicity of five-gallon containers;

FIG. 5 is a close-up view of leaves and a peduncle showing the striping of this new variety;

FIG. 6 shows the height of a stock of this new variety in relation to a yardstick; and

FIG. 7 shows two leaves of this new variety in relation to a ruler.

### DETAILED BOTANICAL DESCRIPTION

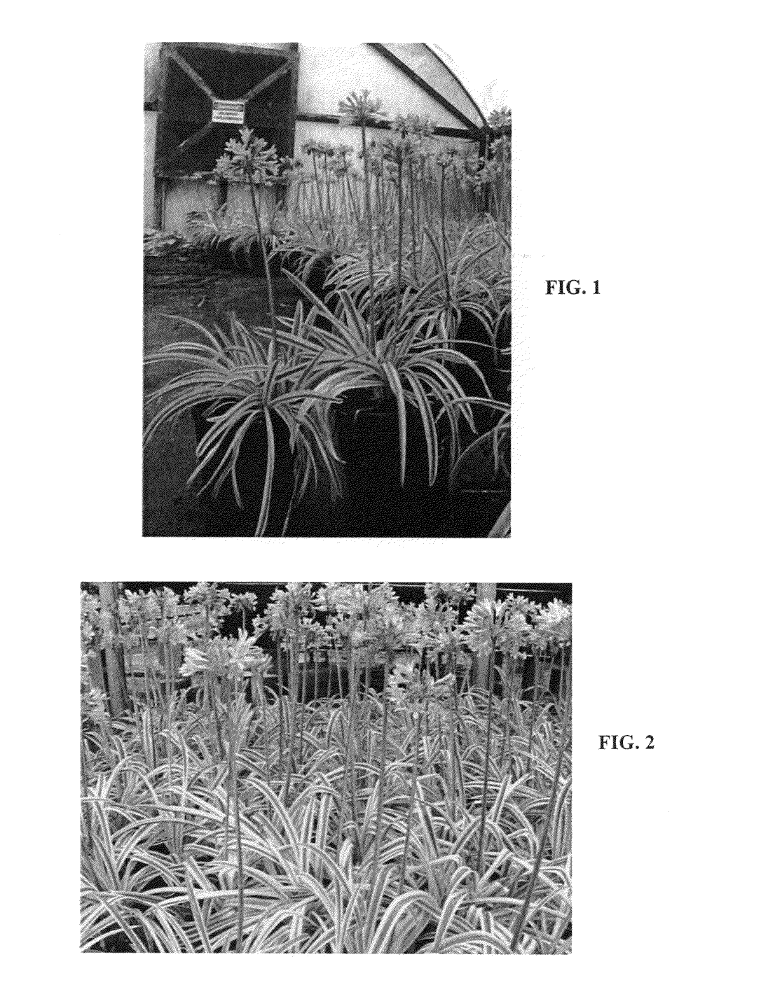

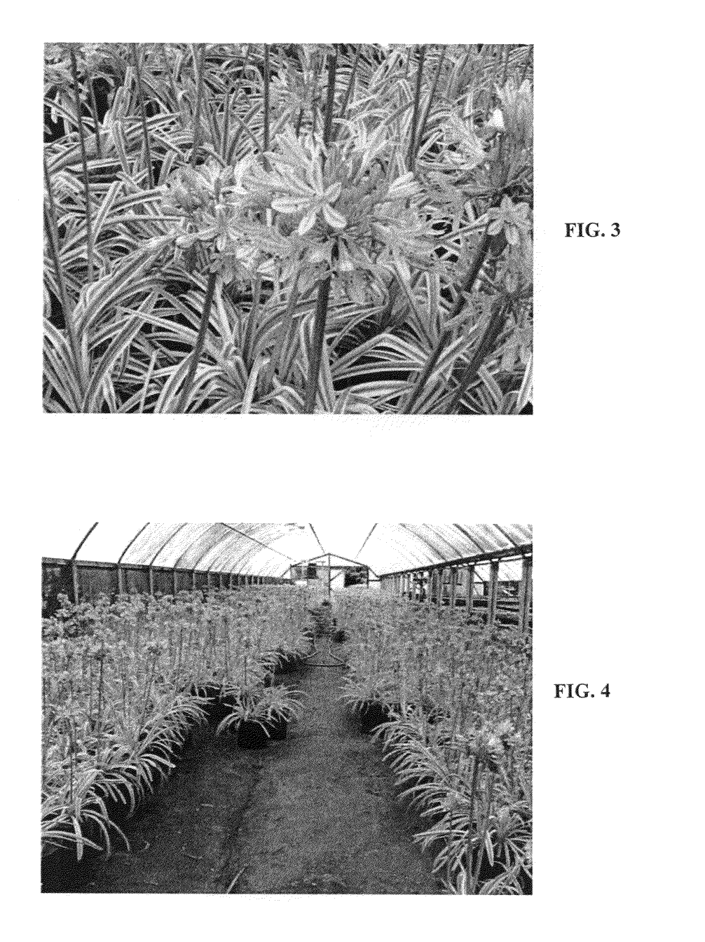

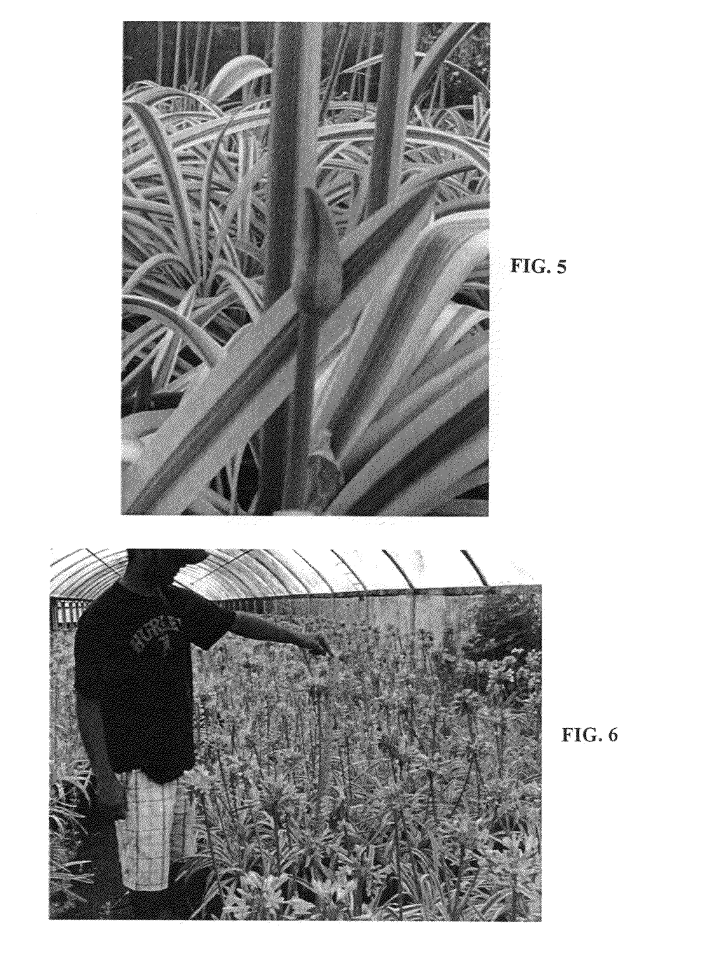

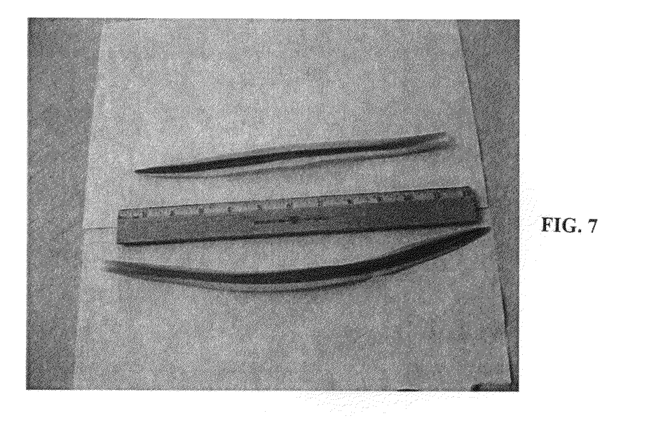

The following is a detailed description of the new Agapanthus africanus cv. Ponto's Queen of the Nile, the color terminology being from the Pantone color chart.
Foliage: Agapanthus africanus cv. Ponto's Queen of the Nile has the same general growth habit of the parent plant Agapanthus africanus cv. Queen Ann. The leaves are eleven to thirteen inches (27.9 to 33.02 cm) in length (FIG. 7). The leaves grow upward and then cascade to a mound of twenty six to thirty one inches (66.04 to 76.20 cm) across, as shown in FIGS. 1 and 4. The peduncle reaches twenty three to thirty one inches (58.42 to 78.74 cm) in height, as shown in FIGS. 1, 2, and 6, producing an umbel flower cluster with fifty to seventy five tubular flowers (FIGS. 2 and 3). The new Agapanthus africanus cv. Ponto's Queen of the Nile has a leaf length of eleven to thirteen inches (27.94 to 33.02 cm) and has a linear border of one quarter inch (0.635 cm) wide of yellow color 101-C tapering to the apex, as shown in FIGS. 5 and 7. The center of the leaf has a green stripe of color 574-C one half inch (1.27 cm) in width, tapering to the apex. The leaves grow upward and then cascade. The striping continues through the entire length of the leaf. The peduncle is green in color 574-C with a yellow stripe of color 101-C. See FIGS. 1, 2, 3, and 5.
Inflorescence: The flowers are produced from a peduncle of twenty three to thirty one inches (58.42 to 78.74 cm) in length (FIGS. 1, 2, and 6). The blooms are a cluster with fifty to seventy five tubular flowers, as shown in FIGS. 2 and 3. The flowers are lavender blue in color 2708-C with a darker lavender stripe through each petal of color 2716-C. See FIGS. 1-3.

What is claimed is:

1. A new, distinctive and different selection of Agapanthus africanus as substantially shown and described herein, that is characterized by a yellow gold leaf with a green stripe through the middle, and a green peduncle with a yellow linear stripe.
